(19) United States
(12) Patent Application Publication
(10) Pub. No.: US 2017/0173285 A1
(43) Pub. Date: Jun. 22, 2017

(54) Fire Safety Oxygen Mask

(71) Applicant: William C. Culp, JR., Holland, TX (US)

(72) Inventor: William C. Culp, JR., Holland, TX (US)

(73) Assignee: Scott & White Healthcare, Temple, TX (US)

(21) Appl. No.: 15/385,271

(22) Filed: Dec. 20, 2016

Related U.S. Application Data
(60) Provisional application No. 62/270,764, filed on Dec. 22, 2015.

(51) Int. Cl. A61M 16/00; A61M 16/08; A61M 16/01; A61M 16/06

(52) U.S. Cl. CPC A61M 16/0087; A61M 16/06; A61M 16/0875; A61M 16/085; A61M 16/01; A61M 2202/0208

(57) ABSTRACT

Provided herein are fire safety face masks and systems comprising the same for displacing excessive oxygen away from a surgical area. The face mask comprises a body portion, an exhalation conduit fluidly connected to the body portion and an in-flow tubing in a fluidly connecting relationship with the body portion. The fire safety face mask may further comprise a gas sampling port disposed on the body portion to facilitate monitoring of inspired and exhaled gases via a multi-gas analyzer. Also provided are methods for preventing fires in an operating theater during a surgical procedure utilizing the fire safety masks and systems disclosed herein.

### CROSS-REFERENCE TO RELATED APPLICATION

This non-provisional application claims benefit of priority under 35 U.S.C. §119(e) of pending provisional application U.S. Ser. No. 62/270,764, filed Dec. 22, 2015, the entirety of which is hereby incorporated by reference.

### BACKGROUND OF THE INVENTION

Field of the invention

The present invention generally relates to the field of medical devices. More specifically, the present invention relates to a fire safety oxygen mask for effectively preventing fires when operating room personnel use supplemental oxygen.

Description of the Related Art

Operating room fires have been deemed as one of the top safety hazards for hospitals by various national safety organizations, including the Emergency Care Research Institute, the Anesthesia Patient Safety Foundation, and The Joint Commission. Every year, over 600 operating room fires occur during surgical procedures in the United States, leading to severe patient injury, disability, and death. Generally, operating room fires occur when the three components of the fire triad, fuel, an oxidizer, and a source of ignition, are brought together. Fuel sources are commonplace in an operating room, including surgical drapes, sponges, dressings, and alcohol based skin antiseptic solutions. Electrical surgical units or lasers often provide the ignition source. Oxygen, known as one of the most common oxidizers, is frequently supplemented by anesthesiologists to prevent patient hypoxemia.

Open supplemental oxygen devices such as the simple face mask and nasal cannula continuously vent oxygen into the atmosphere immediately surrounding the patient. Additionally, patients typically exhale excess oxygen, as well, resulting in elevated oxygen content especially around the nose and mouth area. As a result, oxygen can collect or pool underneath surgical drapes, forming a localized region of dangerously high oxygen that may contribute to operating room fires. Excessive oxygen in the operating room drastically increases the likelihood of fire and accelerates the spreading of fire. Hence, surgical procedures on the head, neck, and upper torso are at increased risk of fire.

Efforts to prevent operating room fires require effective removal of at least one of the components of the fire triad. Fuel sources are ubiquitous and supplemental oxygen is required to maintain life in many patients while undergoing anesthesia. A triggering agent (an ignition source) such as the electrosurgical unit is absolutely needed by the surgeon for most operations. In spite of aggressive safety campaigns focused on educating doctors and nurses about fire risk and prevention, fire incidents continue to occur.

Therefore, there is a recognized need in the art for a fire safety face mask that displaces excessive supplemental oxygen away from a surgical area so as to effectively prevent operating room fires. Particularly, the prior art is deficient in this aspect. The present invention fulfills this long-standing need and desire in the art.

### SUMMARY OF THE INVENTION

The present invention is directed to a fire safety face mask. The face mask comprises a body portion which is a modified face mask. An exhalation conduit is in fluid connection with the body portion. An in-flow tubing is in a fluidly connecting relationship with the body portion.

The present invention also is directed to a method for preventing a fire in an operating theater during a surgical procedure on a patient. The method comprises connecting a source of oxygen to the in-flow tubing on the fire safety face mask as described herein secured to the patient. A distal end of the exhalation conduit is positioned outside of the surgical area. Oxygen is flowed through the in-flow tubing to the patient. Oxygen not inspired by the patient is displaced through the distal end of the exhalation conduit.

The present invention is directed further to a system for preventing a fire during a surgical procedure on a patient in an operating theater. The system comprises the fire safety face mask as described herein and an oxygen source fluidly connectable to the in-flow tubing.

The present invention is directed further still to a fire prevention method during a surgical procedure on a patient in an operating theater. The method comprises flowing oxygen from the oxygen source comprising the system described herein through the in-flow tubing to the fire safety mask on the patient. An excess of the oxygen in the face mask is displaced through the exhalation conduit away from a surgical area in the operating theater.

The present invention is directed further still to a fire safety face mask for displacing an excess of oxygen away from a surgical area. The face mask comprises a body portion comprising a ventless face mask. An exhalation conduit is fluidly connected to the body portion via a swivel connector. An in-flow tubing is disposed within the exhalation conduit in a fluidly connecting relationship with the body portion. A gas sampling port disposed on the body portion.

### BRIEF DESCRIPTION OF THE DRAWINGS

So that the matter in which the above-recited features, advantages and objects of the invention, as well as others that will become clear, are attained and can be understood in detail, more particular descriptions of the invention briefly summarized above may be by reference to certain embodiments thereof that are illustrated in the appended drawings. These drawings form a part of the specification. It is to be noted, however, that the appended drawings illustrate preferred embodiments of the invention and therefore are not to be considered limiting in their scope.

FIG. 1 is a front view of the fire safety mask that comprises a face mask body, face mask straps, oxygen in-flow tubing, an exhalation conduit, a swivel connector and a gas sampling outlet.

FIG. 2 is a side view of the fire safety mask that comprises a face mask body, face mask straps, oxygen in-flow tubing, an exhalation conduit, a swivel connector and a gas sampling outlet.

FIG. 3 depicts a system for preventing a fire during a surgical procedure in an operating theater comprising the fire safety face mask and an oxygen source.

### DETAILED DESCRIPTION OF THE INVENTION

As used herein in the specification, “a” or “an” may mean one or more. As used herein in the claim(s), when used in conjunction with the word “comprising”, the words “a” or “an” may mean one or more than one.

As used herein “another” or “other” may mean at least a second or more of the same or different claim element or components thereof. Similarly, the word “or” is intended to include “and” unless the context clearly indicates otherwise. “Comprise” means “include.”

As used herein, the term “distal” refers to a direction away from the surgical area in an operating room or operating theater.

As used herein, the term “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

In one embodiment of the present invention, there is provided fire safety face mask, comprising a body portion comprising a face mask; an exhalation conduit in fluid connection with said body portion; and an in-flow tubing in a fluidly connecting relationship with said body portion. Further to this embodiment, the fire safety face mask comprises a gas sampling port disposed on the body portion thereof.

In both embodiments, the exhalation conduit may have an airtight connection with the body portion. Also in both embodiments, the exhalation conduit may be fluidly connected to the body portion via a swivel connector. In addition, the exhalation conduit may be about 21 inches to about 25 inches in length. Furthermore, the exhalation conduit may be about 0.5 inches to about 1 inch in diameter. Further still, the exhalation conduit may be expandable and flexible.

In one aspect of these embodiments, the in-flow tubing may be disposed within the exhalation conduit. In another alternative aspect, the in-flow tubing may be fluidly connected to an exterior of the exhalation conduit. In this embodiment, the inspired gas arises from the oxygen in-flow tubing or the exhalation conduit. In an alternative aspect of the embodiment, the in-flow tubing may be attached directly to the body of the face mask or may be fluidly connected to the body portion.

In another embodiment of the present invention, there is provided a method for preventing a fire in an operating theater during a surgical procedure on a patient comprising connecting a source of oxygen to the in-flow tubing on the fire safety face mask as described supra secured to the patient; positioning a distal end of the exhalation conduit outside of the surgical area; flowing oxygen through the in-flow tubing to the patient; and displacing oxygen not inspired by the patient through the distal end of the exhalation conduit.

In yet another embodiment of the present invention, there is provided a system for preventing a fire during a surgical procedure on a patient in an operating theater comprising the fire safety face mask, as described supra, and an oxygen source fluidly connectable to the in-flow tubing. An exemplary oxygen source may include, but is not limited to, an oxygen tank, a central hospital supply or an anesthesia machine.

In another embodiment of the present invention, there is provided a fire prevention method during a surgical procedure on a patient in an operating theater comprising the steps of flowing oxygen from the oxygen source comprising the system described supra through the in-flow tubing to the fire safety mask on the patient; and displacing an excess of the oxygen in the face mask through the exhalation conduit away from the surgical area in the operating theater. In this embodiment, the flowing step may comprise connecting the in-flow tubing comprising the face mask to the oxygen source; securing the face mask around nose and mouth areas of the patient; and starting the oxygen flow. Also in this embodiment, the displacing step may comprise placing a distal end of the exhalation conduit away from a surgical area prior to flowing the oxygen.

In another embodiment of the present invention, there is provided a fire safety face mask for displacing an excess of oxygen away from a surgical area, comprising a body portion comprising a ventless face mask; an exhalation conduit fluidly connected to the body portion via a swivel connector; an in-flow tubing disposed within the exhalation conduit in a fluidly connecting relationship with the body portion; and a gas sampling port disposed on the body portion.

In this embodiment, the exhalation conduit may have an airtight connection with the body portion. Also in this embodiment, the exhalation conduit may be about 21 inches to about 25 inches in length such that the excess of oxygen is displaced away from the surgical area. In addition, the exhalation conduit may be about 0.5 inches to about 1 inch in diameter. Furthermore, the exhalation conduit may be expandable and flexible.

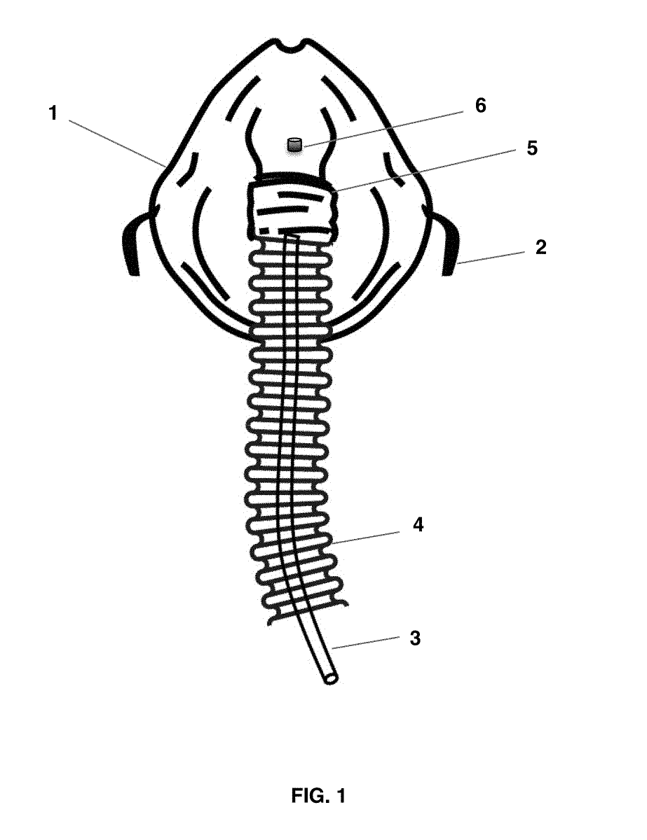

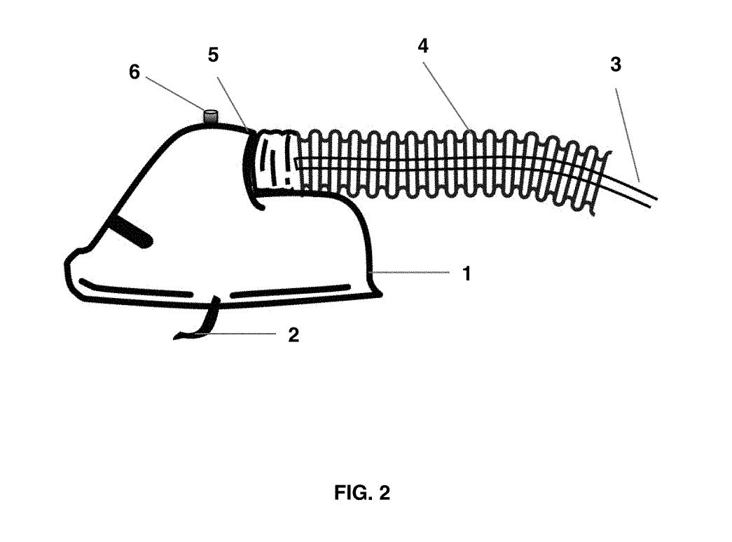

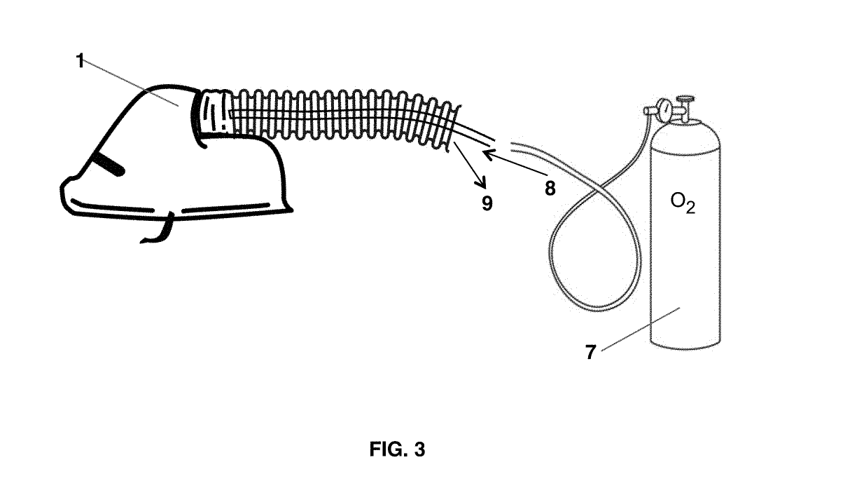

Provided herein are fire safety face masks, systems and methods for effectively preventing an operating room or operating theater fire by displacing excess supplemental oxygen delivered to a patient away from the surgical area. FIGS. 1 and 2 depict the invention in a frontal and a profile view. The fire safety face mask comprises a standard simple oxygen face mask body 1. In a traditional simple face mask, a few openings are disposed on the mask body allowing a patient's exhalation to be vented directly to the atmosphere around the patient's face. The fire safety mask provided herein has eliminated such openings to prevent oxygen venting into the operating area. The mask is secured to the patient's face via a face mask strap 2 in a manner to prevent inspired and expired gases from leaking from the mask. A source of oxygen gas (see FIG. 3), such as, but not limited to, oxygen from a tank, oxygen piped from a central hospital supply or an anesthesia machine, is connected to the face mask via an oxygen in-flow tubing 3, which may be disposed within the interior of the exhalation conduit 4 or, alternatively, may be attached directly to the side of the exhalation conduit 4 or may be connected directly to the face mask body 1.

The exhalation conduit 4 is attached to, connected to or depends from the main body of the face mask where excess supplemental oxygen and other expired gases are directed away from the patient and the surgical area. Preferably, the connections between the exhalation conduit and the body portion of the face mask and between the oxygen in-flow tubing and the body portion are both airtight. The exhalation conduit 4 comprises an expandable, flexible tubing with an approximate diameter of 1″. The exhalation conduit is preferably about 21 inches to about 25 inches in length, thus providing an adequate distance in which to remove oxygen away from the head and upper torso of the patient while minimizing airway resistance. With this design, all inspired gases arise either directly from the oxygen in-flow tubing 3 and/or through the exhalation conduit 4. Optionally, a swivel connector 5 is utilized to attach the exhalation conduit 4 to the face mask body 1, enabling convenient positioning of the exhalation conduit 4. Also, optionally, a gas sampling outlet 6 may be placed on the face mask body 1 to facilitate monitoring of inspired and exhaled gases via a multi-gas analyzer.

FIG. 3 depicts a system comprising the fire safety fire mask and an oxygen source 7 for preventing a fire during a surgical procedure in an operating theater. The oxygen source provides the inspired gas 8, for example, oxygen or oxygen enriched air, to the face mask through in-flow tubing. The excess oxygen not inspired by the patient and the expired gases 9 from the patient are displaced or flowed away from the surgical area via the exhalation conduit.

The present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention.

What is claimed is:

1. A fire safety face mask, comprising:
a body portion comprising a face mask;
an exhalation conduit in fluid connection with said body portion; and
an in-flow tubing in a fluidly connecting relationship with said body portion.

2. The fire safety face mask of claim 1, further comprising a gas sampling port disposed on the body portion thereof.

3. The fire safety face mask of claim 1, wherein said exhalation conduit has an airtight connection with the body portion.

4. The fire safety face mask of claim 1, wherein said exhalation conduit is fluidly connected to the body portion via a swivel connector.

5. The fire safety face mask of claim 1, wherein said exhalation conduit is about 21 inches to about 25 inches in length.

6. The fire safety face mask of claim1, wherein said exhalation conduit is about 0.5 inches to about 1 inch in diameter.

7. The fire safety face mask of claim 1, wherein said exhalation conduit is expandable and flexible.

8. The fire safety face mask of claim 1, wherein said in-flow tubing is disposed within the exhalation conduit.

9. The fire safety face mask of claim 1, wherein said in-flow tubing is fluidly connected to an exterior of the exhalation conduit or is fluidly connected to the body portion.

10. A method for preventing a fire in an operating theater during a surgical procedure on a patient, comprising the steps of:
connecting a source of oxygen to the in-flow tubing on the fire safety face mask of claim 1 secured to the patient;
positioning a distal end of the exhalation conduit outside of the surgical area;
flowing oxygen through the in-flow tubing to the patient; and
displacing oxygen not inspired by the patient through the distal end of the exhalation conduit.

11. A system for preventing a fire during a surgical procedure on a patient in an operating theater, comprising:
the fire safety face mask of claim 1; and
an oxygen source fluidly connectable to the in-flow tubing.

12. The system of claim 11, wherein said oxygen source is an oxygen tank, a central hospital supply or an anesthesia machine.

13. A fire prevention method during a surgical procedure on a patient in an operating theater, comprising the steps of:
flowing oxygen from the oxygen source comprising the system of claim 11 through the in-flow tubing to the fire safety mask on the patient; and
displacing an excess of the oxygen in the face mask through the exhalation conduit away from a surgical area in the operating theater.

14. The fire prevention method of claim 13, wherein the flowing step comprises:
connecting the in-flow tubing comprising the face mask to the oxygen source;
securing the face mask around nose and mouth areas of the patient; and
starting the oxygen flow.

15. The fire prevention method of claim 13, wherein the displacing step comprises:
placing a distal end of the exhalation conduit away from the surgical area prior to flowing the oxygen.

16. A fire safety face mask for displacing an excess of oxygen away from a surgical area, comprising:
a body portion comprising a ventless face mask;
an exhalation conduit fluidly connected to said body portion via a swivel connector;
an in-flow tubing disposed within said exhalation conduit in a fluidly connecting relationship with said body portion; and
a gas sampling port disposed on the body portion.

17. The fire safety face mask of claim 16, wherein the exhalation conduit has an airtight connection with the body portion.

18. The fire safety face mask of claim 16, wherein said exhalation conduit is about 21 inches to about 25 inches in length such that the excess of oxygen is displaced away from the surgical area.

19. The fire safety face mask of claim 16, wherein said exhalation conduit is about 0.5 inches to about 1 inch in diameter.

20. The fire safety face mask of claim 16, wherein said exhalation conduit is expandable and flexible.
